(19) United States
(12) Patent Application Publication
(10) Pub. No.: US 2026/0152421 A1
(43) Pub. Date: Jun. 4, 2026

(54) DEVICE FOR MEASURING THE ALKALINITY OF A LIQUID PRESENT IN AN EXTERNAL RESERVOIR AND PROCESS FOR MEASURING THE ALKALINITY OF A LIQUID PRESENT IN AN EXTERNAL RESERVOIR

(71) Applicant: AQUARINO COMERCIO AUTOMAÇÃO E SERVIÇOS LTDA, Rio de Janeiro (BR)

(72) Inventors: LAERCIO DE MENDONÇA FURTADO, Jr., Rio de Janeiro (BR); ISRAEL LINS ALBUQUERQUE, Rio de Janeiro (BR)

(21) Appl. No.: 19/256,877

(22) Filed: Jul. 1, 2025

(30) Foreign Application Priority Data
Oct. 31, 2024 (BR) 102024022808-1

(51) Int. Cl. C02F 1/66; B01F 23/237; B01F 101/00; C02F 103/00

(52) U.S. Cl. CPC C02F 1/66; B01F 23/237611; B01F 2101/305; C02F 2103/007; C02F 2209/006; C02F 2209/06; C02F 2209/07; C02F 2209/24

(57) ABSTRACT

The present invention relates to a device (1) for measuring the alkalinity of a liquid (41) present in an external reservoir (40). More particularly, the proposed device (1) is used in the aquarium sector for monitoring water quality (41) in marine aquariums (40), performing the mensuration and automatic correction of the alkalinity of this water (41), especially in aquariums (40) that have coral reefs and other sensitive organisms present in saltwater aquariums. In addition, the present invention also proposes a process for measuring the alkalinity of a liquid (41) in an external reservoir (40). In particular, the proposed device (1) and process are characterized in that they comprise a control unit (70) capable of determining the alkalinity of an external liquid (41) present in an external reservoir (40) to the mensuration device (1) by using a CO2SYS method in conjunction with information previously stored in said control unit (70).

### FIELD OF THE INVENTION

The present invention relates to a device for measuring the alkalinity of a liquid present in an external reservoir. More particularly, the proposed device is used in the aquarium sector for monitoring water quality in marine aquariums, performing the mensuration and automatic correction of the alkalinity of this water, especially in aquariums that have coral reefs and other sensitive organisms present in saltwater aquariums. In addition, the present invention also proposes a process for measuring the alkalinity of a liquid in an external reservoir.

### STATE OF THE ART

Maintaining water alkalinity in marine aquariums, especially in systems that house corals, presents a significant challenge due to the need for constant and accurate monitoring of hydrogen potential (pH) and carbonate buffering (KH). Alkalinity is a measure of anions that readily bind to protons to prevent acidification. It turns out that the most common anions that allow water to resist acidification are carbonates and bicarbonates, so alkalinity measurements usually equate to KH measurements.

Adequate alkalinity is crucial to the health of aquatic organisms as it influences essential biological processes such as coral calcification and ecosystem stability, indicating how susceptible water is to changes in pH. In other words, the higher the alkalinity, the more stable the pH of an aquarium will be.

### Technical Problem

Conventionally, the measurement of water alkalinity in aquariums has been based on colorimetric methods and titration systems, which, although effective, have several drawbacks. In these conventional methods, in an already widely known way, a chemical reagent is used in a water sample and depending on the amount of reagent used the water sample changes color, indicating the alkalinity value of that sample.

In this sense, the use of chemical reagents to measure the alkalinity of water in aquariums can be expensive and generate residues harmful to the environment, due to the high frequency required to perform this test. In addition, these methods require manual testing, and when equipment based on the concept of colorimetric methods is used, with the frequent use of chemical reagents, frequent calibrations are necessary, making the process more susceptible to human errors and inconsistencies in results.

### SUMMARY OF THE INVENTION

In order to solve these drawbacks, the present invention proposes a device for measuring the alkalinity of an external liquid present in an external reservoir, comprising a reference reservoir and a transfer reservoir cooperating with a measurement reservoir, at least one pH sensor being arranged inside said measurement reservoir.

In addition, the proposed mensuration device comprises a first pumping device capable of transferring a liquid between the reference reservoir and the measurement reservoir, a second pumping device capable of transferring a liquid between the transfer reservoir and the measurement reservoir, and a third pumping device capable of transferring a liquid between the transfer reservoir and a reservoir external to the mensuration device.

An air pump is used next to the mensuration device to inject air into the inside of the measurement reservoir and the inside of the transfer reservoir.

In particular, the proposed mensuration device is characterized in that it comprises a control unit cooperating with the pH sensor of the measurement reservoir, so as to receive and store the signals from said pH sensor. The control unit also cooperates with the pumping devices and the air pump in such a way as to enable the actuation of said pumping devices and said air pump by means of pre-programmed configurations.

In this way, the control unit is able to determine the alkalinity of an external liquid present in the external reservoir by using a CO2SYS method in conjunction with information previously stored in said control unit.

Furthermore, the present invention also proposes a process for measuring the alkalinity of a liquid present in an external reservoir, comprising an alkalinity mensuration device cooperating with said external reservoir in which said liquid is located.

In particular, the proposed process is characterized in that the mensuration device comprises a control unit capable of performing a calibration process, in which the alkalinity of a reference liquid present in a reference reservoir of the mensuration device is determined, and a reading process, in which the alkalinity of an external liquid present in a reservoir external to the mensuration device is determined by using a CO2SYS method in conjunction with information previously stored in said control unit.

For information, the CO2SYS method is a method already widely used in several practical applications, such as climate change monitoring, water resources management and ecosystem modeling.

The CO2SYS method was initially launched in 1998 as a DOS interface program and has been developed by researchers to the present day. This method is a family of software that calculates chemical balances for species and parameters of aquatic inorganic carbon. Its main function is to use any two of the four central parameters of the inorganic carbon system (pH, alkalinity, dissolved inorganic carbon, and carbon dioxide partial pressure) to calculate various chemical properties of the system. These programs are widely used by oceanographers and limnologists to understand and predict chemical balances in natural waters.

The original CO2SYS program for DOS was created by Ernie Lewis and Doug Wallace. This was translated to MATLAB by Denis Pierrot and further optimized by Steven van Heuven. Jim Orr and co-authors added additional sets of equilibrium constants and implemented error propagation in a separate program. The latest version of MATLAB was translated into Python as PyCO2SYS by Matthew Humphreys, benefiting enormously from all this previous work. Modifications and additions to the PyCO2SYS code and documentation were made by Matthew Humphreys, Luke Gregor, Daniel Sandborn, and Abigail Schiller.

The CO2SYS method calculates the xCO2 dissolved in water for a known alkalinity and pH. It is also possible based on the CO2SYS method to obtain an unknown alkalinity based on a known xCO2 factor dissolved in water and pH. This mathematical model of marine carbonate system behavior as a function of alkalinity, pH and xCO2 is the foundation of automation processes performed by the device and process proposed in the present invention.

The model, when fed with high-quality data such as pH, pCO2, temperature, and salinity, can provide reliable estimates of the concentrations of the different forms of inorganic carbon in addition to the carbonate saturation state in relation to calcite and aragonite. The use of newly adjusted equilibrium constants and the ability to incorporate empirical data make CO2SYS especially robust. In addition, continuous validation of the model through direct measurements in the field strengthens confidence in its predictions.

In summary, the CO2SYS model is a fundamental opensource tool in research on the interactions between inorganic carbon and aquatic ecosystems. With its solid scientific foundations, accurate forecasts and broad applications, it becomes an indispensable resource for scientists and managers seeking to understand and mitigate the impacts of climate change and ocean acidification on aquatic environments. Thus, the present invention aims to propose a device and a process that can make effective use of the already known CO2SYS method.

### Advantages

Advantageously, with the use of the CO2SYS method together with the device and the process to measure the alkalinity of a liquid in a proposed external reservoir, it is possible to achieve an automated sensing system, through the control unit equipped with pre-programmed configurations, which does not depend on the frequent use of chemical reagents, in order to reduce the operating costs associated with the purchase and disposal of these reagents. In addition, process automation eliminates the need for frequent manual interventions, resulting in more accurate and reliable measurements of water alkalinity in aquariums.

Advantageously, the control unit of the proposed mensuration device may be programmed to perform continuous measurement and correction of the alkalinity of an aquarium. This automation achieved with the proposed device and process allows for better remote monitoring and more efficient management of an aquarium's water parameters, which can transform the experience of aquarists at both amateur and professional levels. In this context, the implementation of the proposed solution not only improves the health of marine organisms, but also represents a solution for aquarists seeking to ensure the sustainability of their aquatic environments. Therefore, the use of the proposed device and process is not only a technical choice, but a declaration of commitment to the health of marine ecosystems and economic efficiency in aquarium maintenance.

Advantageously, based on the CO2SYS mathematical model, which calculates the water properties in marine carbonate systems from two parameters, it is possible to carry out a continuous, accurate and reliable process of measuring and correcting the alkalinity of aquarium water.

### BRIEF DESCRIPTION OF THE DRAWINGS

The invention will be better understood with the following detailed description, which will be better understood with the aid of the drawings, namely:

FIG. 1 shows a front view of a mensuration device (1) as proposed by the present invention.

FIG. 2 shows a perspective view of the mensuration device (1) with emphasis on pumping devices (51,52,53).

FIG. 3 shows a side view of the mensuration device (1) with emphasis on the connection ports (90).

FIG. 4 shows an open rear view of the mensuration device (1) with emphasis on the internal components of said device (1).

FIG. 5 shows a top view of the mensuration device (1) with emphasis on the interconnections between the reservoirs (10,20,30) and the pumping devices (51,52,53) of said device (1).

### DESCRIPTION OF THE INVENTION

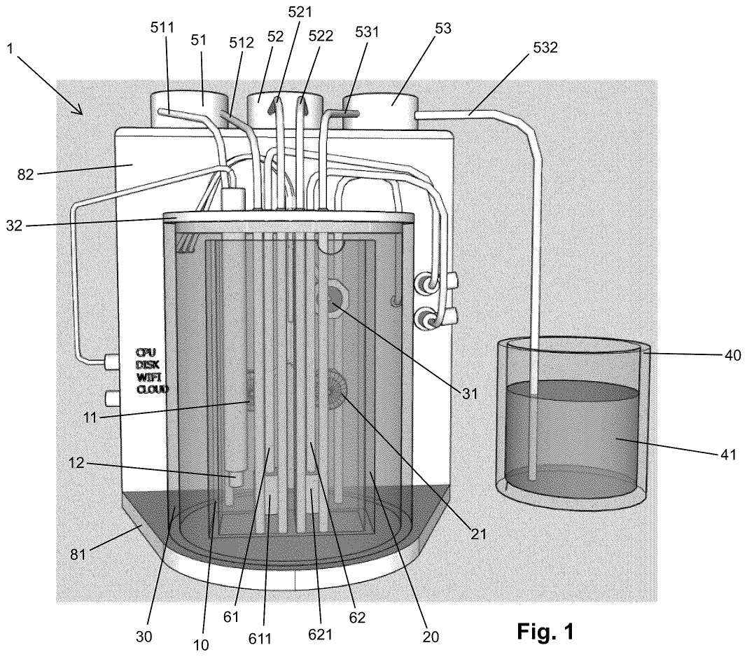

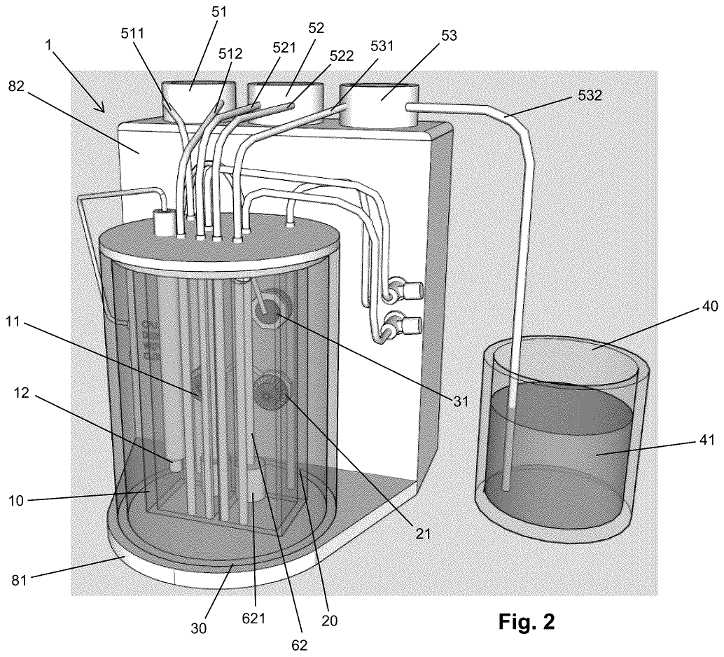

As can be seen in FIGS. 1 to 3, the present invention proposes a device (1) for measuring the alkalinity of an external liquid (41) present in an external reservoir (40) to said device (1), comprising a reference reservoir (30) and a transfer reservoir (20) cooperating with a measurement reservoir (10), at least one pH sensor (12) being arranged inside said measurement reservoir (10).

Preferably, the proposed mensuration device (1) comprises a level sensor (11,21,31) arranged inside each of said reservoirs (10,20,30) intended to facilitate the automation of the operation of said device (1). Alternatively, it is possible to replace the functionalities provided by the level sensors (11,21,31) with other components that allow the automation of the operation of the mensuration device (1), such as the filling time of the reservoirs (10,20,30).

Preferably, the reservoirs (10,20,30) are containers made of acrylic, the reference reservoir (30) comprising an airtight lid (32), and the level sensors (11,21,31) are widely known optical infrared level sensors, and the pH sensor (12) is a pH probe, also widely known.

In the shown configuration, a single pH sensor (12) is used to ensure the same pH measurement conditions during the processes that will be better described below, ensuring greater reliability in the results. However, it is possible to use more than one pH sensor in the different reservoirs (10,20,30), but the same pH measurement conditions must be guaranteed in order to obtain reliable results.

Furthermore, the proposed mensuration device (1) comprises a first pumping device (51) able to transfer a liquid between the reference reservoir (30) and the measurement reservoir (10), a second pumping device (52) able to transfer a liquid between the transfer reservoir (20) and the measurement reservoir (10) and a third pumping device (53) able to transfer a liquid between the transfer reservoir (20) and the external reservoir (40) to said mensuration device (1).

Preferably, the pumping devices (51,52,53) are peristaltic pumps responsible for transporting liquids between the reservoirs (10,20,30) of the mensuration device (1) and between the external reservoir (40) and said mensuration device (1). As is already widely known, the pumping devices (51,52,53) of the peristaltic pump type are provided with operation openings in which a liquid is transported according to the direction of operation of said pumping devices (51,52,53). In other words, during the operation of the pumping devices (51,52,53), a liquid is transported from one operation opening towards the other operation opening and vice versa, depending on the direction of operation of said pumping devices (51,52,53).

Furthermore, the mensuration device (1) comprises an electromagnetic air pump (60) capable of injecting air into the measurement reservoir (10) and into the transfer reservoir (20).

Preferably, the air pump (60) simultaneously injects air into both the interior of the measurement reservoir (10) and the interior of the transfer reservoir (20).

In particular, the proposed mensuration device (1) is characterized in that it comprises a control unit (70) cooperating with the level sensors (21,31,41) of the reservoirs (10,20,30) and with the pH sensor (12) of the measurement reservoir (10), in order to receive and store the signals from said level sensors (21,31,41) and from said pH sensor (12). Furthermore, the proposed control unit (70) cooperates with the pumping devices (51,52,53) and with the air pump (60), so that said mensuration device (1) is able to drive said pumping devices (51,52,53) and said air pump (60) from pre-programmed configurations.

More specifically, the control unit (70) is able to determine the alkalinity of an external liquid (41) present in the external reservoir (40) by using a CO2SYS method in conjunction with information previously stored in said control unit (70).

Preferably, the control unit (70) is an electronic board capable of receiving and storing the signals of all the sensors (11,21,31,12) arranged in the mensuration device (1), and of controlling all the devices (51,52,53) arranged in said mensuration device (1).

Preferably, the mensuration device (1) comprises a structure (80) on which the reservoirs (10,20,30), the pumping devices (51,52,53), the air pump (60) and the control unit (70) are mounted.

Preferably, both the measurement reservoir (10) and the transfer reservoir (20) are arranged inside the reference reservoir (30). In the shown embodiment, the measurement reservoir (10) and the transfer reservoir (20) have a height that does not touch the inside of the cap (32) of the reference reservoir (30) in order to allow an overflow of water from the interior of said measurement reservoir (10) and said transfer reservoir (20) to the interior of the reference reservoir (30).

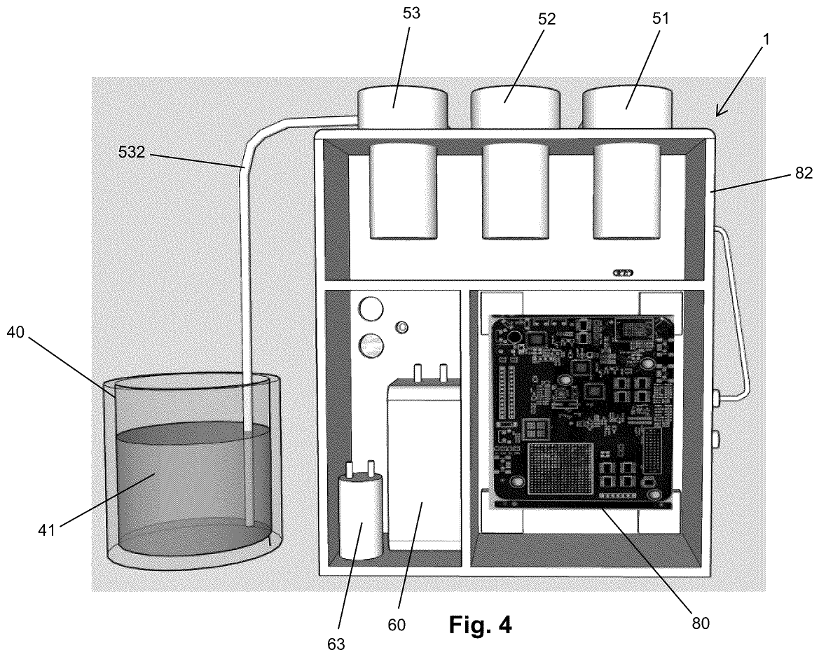

In the shown embodiment, the structure (80) of the mensuration device (1) comprises a horizontal base (81) on which the reference reservoir (30) is mounted, with the respective measurement (10) and transfer (20) reservoirs mounted within. As can be best seen in FIG. 4, the structure (80) of the mensuration device (1) also comprises a vertical enclosure (82) within which the pumping devices (51,52,53), the air pump (60) and the control unit (70) are mounted. It is noted that an upper portion of the pumping devices (51,52,53) protrudes out of the vertical enclosure (82), wherein it is in this upper portion that hoses are installed in order to allow the transfer of liquids between the reservoirs (10,20,30).

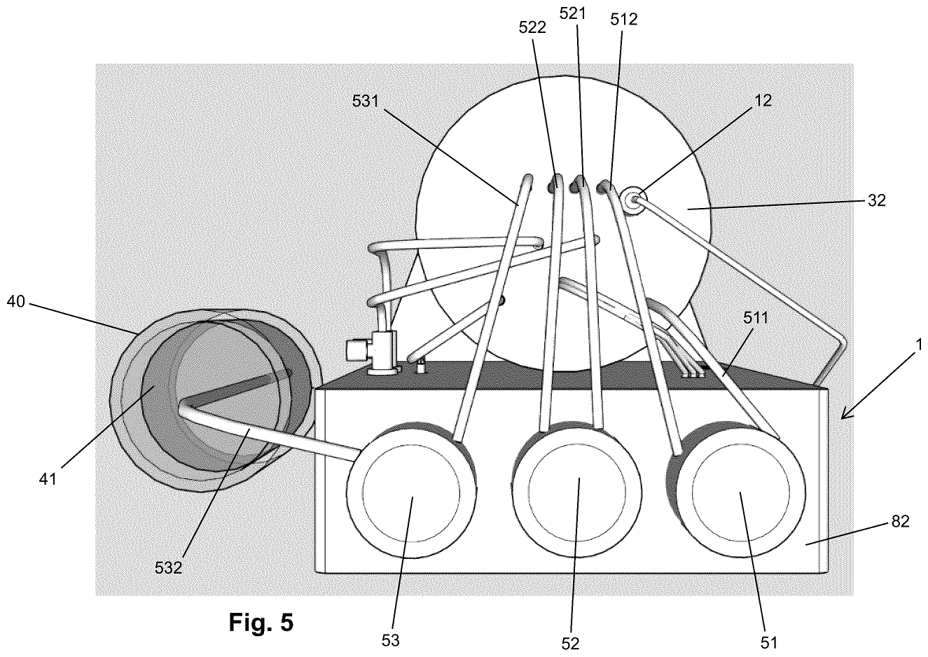

As can be best seen in FIGS. 1 to 5, the first pumping device (51) comprises a first hose (511) with one end connected at one of the operating openings of said first pumping device (51) and with the other end arranged inside the reference reservoir (30), and a second hose (512) with one end connected at the other operating opening of said first pumping device (51) and with the other end arranged inside the measurement reservoir (10).

The second pumping device (52) comprises a first hose (521) with one end connected at one of the operating openings of said second pumping device (52) and with the other end arranged inside the measurement reservoir (10), and a second hose (522) with one end connected at the other operating opening of said second pumping device (52) and with the other end arranged inside the transfer reservoir (20).

The third pumping device (53) comprises a first hose (531) with one end connected at one of the operating openings of said third pumping device (53) and with the other end arranged inside the transfer reservoir (20), and a second hose (532) with one end connected at the other operating opening of said third pumping device (53) and with the other end arranged inside the external reservoir (40).

Preferably, the hoses (511,512,521,522,531) that are arranged inside the reservoirs (10,20,30) of the mensuration device (1) extend to the bottom of the respective reservoirs (10,20,30) to ensure that all the liquid present in said reservoirs (10,20,30) is properly suctioned by the pumping devices (51,52,53) when said mensuration device (1) is in operation.

Preferably, the mensuration device (1) comprises a first porous stone (611) and a second porous stone (621) cooperating with the air pump (60), said first porous stone (611) being arranged inside the measurement reservoir (10) and said second porous stone (621) being arranged inside the transfer reservoir (20).

In the embodiment depicted, a first hose (61) is connected at a first outlet of the air pump (60) and disposed at the bottom of the interior of the measurement reservoir (10) and a second hose (621) is connected at a second outlet of the air pump (60) and disposed at the bottom of the interior of the transfer reservoir (20). Thus, the first porous stone (611) may be mounted on the tip of the first hose (61) of the air pump (60) and be disposed at the bottom of the measurement reservoir (10), and the second porous stone (621) may be mounted on the tip of the second hose (62) of the air pump (60) and be disposed at the bottom of the transfer reservoir (20).

Preferably, the mensuration device (1) comprises a condensation filter (63) connected in series with the air pump (60) responsible for filtering the air suctioned by said air pump (60) at the top of the reference reservoir (30) to be injected into the measurement reservoir (10) and the transfer reservoir (20). Thus, the condensation filter (63) guarantees greater durability of the air pump (60) during its use with the proposed mensuration device (1).

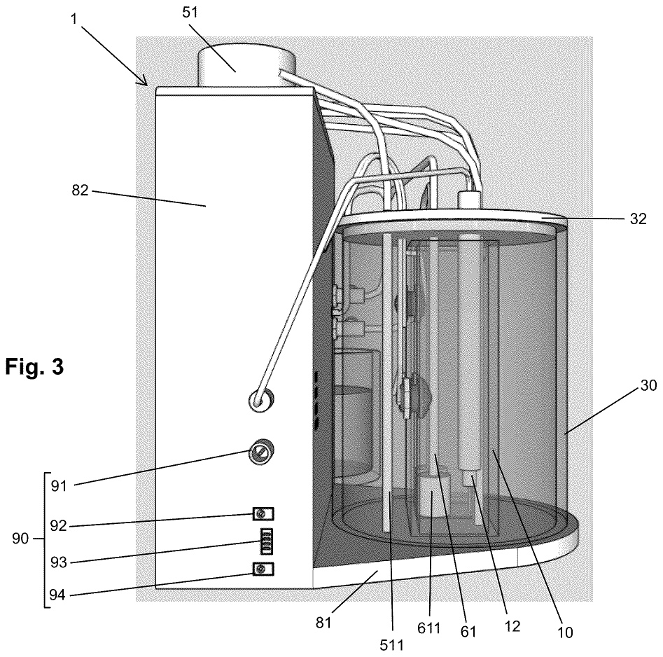

As can be best seen in FIG. 3, the mensuration device (1) comprises at least one connection port (90) cooperating with the control unit (70) intended to connect with a power source and/or an auxiliary device for aquarium control.

Preferably, the mensuration device (1) comprises a plurality of connection ports (90), being a first port (91) for integration with other commercial aquarium controllers, a second port (92) for integration with external dosers, such as for example calcium reactors, a third port (93) for integration with a CO2 solenoid of calcium reactors and a fourth port (94) for the supply of electrical power, such as for example a 12V source.

In an embodiment not shown, the mensuration device (1) comprises a correction device capable of depositing correction products inside the external reservoir (40) upon actuation of the control unit (70). Correction devices already known and available on the market can be used for this action, such as calcium reactors, as long as they are able to connect with the proposed mensuration device (1). Correction products are understood to be any product that can be added to the external liquid (41) present in the external reservoir (40) in order to change its properties.

Preferably, the proposed mensuration device (1) is used to measure the alkalinity of water in aquariums. Thus, the external liquid (41) and external reservoir (40) cited so far in this specification may be, respectively, water from an aquarium.

Thus, the control unit (70) may comprise pre-programmed configurations for performing a filling process and a water emptying process on the mensuration device (1). As will be further detailed below, with the filling process, all reservoirs (10,20,30) of the mensuration device (1) are filled with water and the respective times are stored in the control unit (70). With the stored time information, it is possible to carry out the proper process of emptying the reservoirs (10,20,30) of the mensuration device (1) for its proper transport and storage, as will also be further detailed below.

For the filling process, an action must be triggered in the control unit (70) so that the third pumping device (53) is triggered in the direction of withdrawing water from the external reservoir (40) into the transfer reservoir (20) until the level sensor (21) indicates that the predetermined limit in the control unit (70) for said transfer reservoir (20) has been reached. This time is stored in the control unit (70) to know the time required to collect new water samples in the periodic tests.

At this time, the third pumping device (53) must continue in operation performing the overflow of the water present in the transfer reservoir (20) to the reference reservoir (30) until the level sensor (31) indicates that the predetermined limit in the control unit (70) for said reference reservoir (30) has been reached. Again, this time is stored in the control unit (70) to acknowledge the required filling time of the reference reservoir (30) which will be used for the emptying process when requested by the user.

After filling the reference reservoir (30) the control unit (70) actuates the third pumping device (53) in order to transfer the excess water from the transfer reservoir (20) to the external reservoir (40) until the water is below the limit established by the level sensor (21) of said transfer reservoir (20), at which time the actuation of the third pumping device (53) is interrupted.

With the reference reservoir (30) and the transfer reservoir (20) at the correct levels, the first pumping device (51) is actuated in the direction of transferring water from the reference reservoir (30) to the measurement reservoir (10) until the level sensor (11) indicates that the predetermined limit in the control unit (70) for said measurement reservoir (10) has been reached, at which time the actuation of the first pumping device (51) is interrupted, ending the filling process completely.

In the emptying process, all water from the reservoirs (10,20,30) of the mensuration device (1) is removed, leaving said device (1) empty to be switched off and transported.

For the emptying process, an action must be triggered in the control unit (70) so that the three pumping devices (51,52,53) are driven by the previously known filling time in order to transfer all the water from the reference reservoir (30) to the measurement reservoir (10) by means of the first pumping device (51), which in turn is transferred from the measurement reservoir (10) to the transfer reservoir (20) by means of the second pumping device (52) and which in turn is transferred from the transfer reservoir (20) to the external reservoir (40) by means of the third pumping device (53), thus ending the emptying process.

The present invention also proposes a process for measuring the alkalinity of an external liquid (41) present in an external reservoir (40), comprising a mensuration device (1) of alkalinity cooperating with said external reservoir (40) in which said external liquid (41) is located. In particular, the mensuration device (1) used in the proposed process comprises a control unit (70) able to perform a calibration process, in which the alkalinity of a reference liquid present in a reference reservoir (30) of the mensuration device (1) is determined, and a reading process, in which the alkalinity of an external liquid present in an external reservoir (40) is determined to the mensuration device (1) through the use of a CO2SYS method in conjunction with information previously stored in said control unit (70).

Preferably, the determination of the alkalinity of the reference liquid present in the reference reservoir (30) of the mensuration device (1), in the calibration process, is carried out by using a CO2SYS method in conjunction with information previously stored in said control unit (70), in which the alkalinity of the external liquid present in the external reservoir (40) must first be stored in the control unit (70) by means of a conventional market test that uses reagent and the control unit (70) stores this information.

Preferably, the proposed process is used to measure the alkalinity of water in aquariums.

Thus, preferably, after the initial procedure of informing the alkalinity of a calibration water present in an external reservoir (40), and considering that both the measurement reservoir (10) and the transfer reservoir (20) of the mensuration device (1) are empty, the calibration process of said mensuration device (1) comprises the steps that will be detailed below.

Step of transferring a reference water with unknown alkalinity present in the reference reservoir (30) to the measurement reservoir (10) by means of the first pumping device (51) until the level sensor (11) indicates that the limit of the measurement reservoir (10) has been reached.

Step of transferring, by means of the third pumping device (53) driven by the control unit (70), a sample of the calibration water with known alkalinity present in the external reservoir (40), and informed by the operator of the device (1), to the transfer reservoir (20) until the level sensor (21) indicates that the limit of said transfer reservoir (20) has been reached.

Step of measuring relative pH of the reference water with unknown alkalinity present in the measurement reservoir (10) and storing this information in the control unit (70) for use in later steps.

Step of injecting air into the reference water present in the measurement reservoir (10) and the calibration water present in the transfer reservoir (20) for sufficient time to stabilize the relative pH, and then storing the pH information of the reference water with unknown alkalinity that is in the measurement reservoir (10). In this step, the control unit (70) drives the air pump (60), which collects the air from the top of the reference reservoir (30) and injects through the two porous stones (611,621) that are at the bottom of the measurement (10) and transfer (20) reservoirs, in order to guarantee a uniformity of the gases contained in the water samples present in said reservoirs (10,20), mainly for xCO2, which is one of the parameters, already known, necessary for the use of the CO2SYS method.

Preferably, the control unit (70) of the mensuration device (1) constantly measures the relative pH of the reference water present in the measurement reservoir (10) until it reaches stability in the reading. It is recommended that the maximum time controlled by said control unit (70) for this procedure be 20 minutes for relative pH stabilization. After stabilization of the relative pH, the control unit (70) turns off the air pump (60), waits one minute and reads the relative pH of the reference water that is in the measurement reservoir (10), which now has CO2 levels equivalent to the calibration water that is in the transfer reservoir (20).

At this stage of the step, the control unit (70) performs the final reading of the relative pH of the reference water with unknown alkalinity that is in the measurement reservoir (10), to perform the calculations in later steps to obtain the unknown alkalinity of said reference water that will be stored in the reference reservoir (30).

Step of transferring the reference water with unknown alkalinity from the measurement reservoir (10) to the reference reservoir (30). In this step, control unit (70) drives the first pumping device (51) in the direction of transferring the reference water from the measurement reservoir (10) back to the reference reservoir (30), leaving said measurement reservoir (10) completely dry.

Step of transferring the calibration water with known alkalinity from the transfer reservoir (20) to the measurement reservoir (10). In this step, the control unit (70) drives the second pumping device (52) in the direction of transferring the calibration water with known alkalinity, and informed by the operator of the mensuration device (1), which is in the transfer reservoir (20) to the measurement reservoir (10) and drives again the air pump (60), preferably for 1 minute. Preferably, after 1 minute of aeration process, the control unit (70) turns off the air pump (60), waits for 1 minute for stabilization of the relative pH reading of the calibration water with known alkalinity and stores this information.

Step of using CO2SYS method to calculate the xCO2 for the air that is inside the reservoirs. With the relative pH of the calibration water that had its origin in the external reservoir (40) and the known alkalinity of this calibration water informed by the device operator, the control unit (70) uses the calculations and functions of the CO2SYS method to calculate the xCO2 for the air that is inside the reservoirs (10,20,30) of the mensuration device (1).

Step of using the previously calculated xCO2 together with the relative pH of the reference water present in the reference reservoir (30) to calculate the unknown alkalinity of said reference water by means of the equations and functions of the CO2SYS method and store this information in the control unit (70).

Step of transferring the calibration water from the measurement reservoir (10) to the transfer reservoir (20) by means of the actuation by the control unit (70) of the second pumping device (52) in order to completely transfer said calibration water from the measurement reservoir (10) to the transfer reservoir (20).

To complete the calibration process of the mensuration device (1), the step of transferring the reference water from the reference reservoir (30) to the measurement reservoir (10) is carried out by means of activation by the control unit (70) of the first pumping device (51) in order to transfer said reference water from the reference reservoir (30) to the measurement reservoir (10), keeping the pH sensor (12) submerged.

The calibration process described above finalizes obtaining an unknown alkalinity of the reference water present in the reference reservoir (30) based on a calibration water with known alkalinity informed by the user for the external reservoir (40), using as a basis the CO2 equilibrium and CO2SYS mathematical model.

Due to the reference water being stored in the hermetically sealed reference reservoir (30), its alkalinity remains stable for a long period. It is possible to have a slight change in the alkalinity of the reference water due to the possible presence of microorganisms in said reference water. However, this change in alkalinity can be considered negligible within a period of 90 days. Therefore, considering that the alkalinity can vary from 0 to 14 units of measurement, it is recommended that the calibration process be performed after a certain period, or when the alkalinity of the reference water has a variation of 1 unit of measurement.

Preferably, after the calibration process has been carried out, the mensuration device (1) is able to carry out the reading process by performing the steps that will be detailed below. In the reading process, the control unit (70) keeps stored the alkalinity of the reference water obtained by the calibration process and with this information performs all the processes to obtain the alkalinity of an external water (41) with unknown alkalinity present in an external reservoir (40). Considering that the measurement reservoir (10) and the transfer reservoir (20) are empty, proceed to the following steps for the reading process.

Step of transferring the reference water from the reference reservoir (30) to the measurement reservoir (10), with time control for this operation. For this step, the control unit (70) drives the first pumping device (51) in the direction of transferring the reference water from the reference reservoir (30) to the measurement reservoir (10) until the level sensor (11) indicates that the limit of said measurement reservoir (10) has been reached.

Step of transferring the external water (41) from the external reservoir (40) to the transfer reservoir (20), with time control for this operation. For this step, the control unit (70) drives the third pumping device (53) in the direction of transferring the external water (41) from the external reservoir (40) to the transfer reservoir (20) until the level sensor (21) indicates that the limit of said transfer reservoir (20) has been reached.

Step of measuring the relative pH of the reference water present in the measurement reservoir (10), which has the known alkalinity, and storing this information in the control unit (70) for use in later steps.

Step of injecting air into the reference water present in the measurement reservoir (10) and the external water (41) present in the transfer reservoir (20) for sufficient time to stabilize the relative pH, and then storing the pH information of the reference water with known alkalinity that is in the measurement reservoir (10). In this step, the control unit (70) drives the air pump (60), which collects the air from the top of the reference reservoir (30) and injects through the two porous stones (611.621) that are at the bottom of the measurement (10) and transfer (20) reservoirs, in order to guarantee a uniformity of the gases contained in the water samples present in said reservoirs (10.20), mainly for xCO2, which is one of the parameters, already known, necessary for the use of the CO2SYS method.

Preferably, the control unit (70) of the mensuration device (1) constantly measures the relative pH of the reference water present in the measurement reservoir (10) until it reaches stability in the reading. It is recommended that the maximum time controlled by said control unit (70) for this procedure be 20 minutes for relative pH stabilization. After stabilization of the relative pH, the control unit (70) turns off the air pump (60), waits one minute and reads the relative pH of the reference water that is in the measurement reservoir (10), and which now has CO2 levels equivalent to the external water that is in the transfer reservoir (20).

At this stage of the step, the control unit (70) performs the final reading of the relative pH of the reference water with known alkalinity that is in the measurement reservoir (10), to perform the calculations in later steps to obtain the unknown alkalinity of the external water that is in the transfer reservoir (20).

Step of transferring the reference water with known alkalinity from the measurement reservoir (10) to the reference reservoir (30). In this step, control unit (70) drives the first pumping device (51) in the direction of transferring the reference water from the measurement reservoir (10) back to the reference reservoir (30), leaving said measurement reservoir (10) completely dry.

Step of transferring the external water (41) with unknown alkalinity from the transfer reservoir (20) to the measurement reservoir (10). In this step, the control unit (70) drives the second pumping device (52) in the direction of transferring the external water (410 with unknown alkalinity that is in the transfer reservoir (20) to the measurement reservoir (10) and drives again the air pump (60), preferably for 1 minute. Preferably, after 1 minute of aeration process, the control unit (70) turns off the air pump (60), waits for 1 minute for stabilization of the relative pH reading of the external water (41) with unknown alkalinity and stores this information.

Step of using the CO2SYS method to obtain the alkalinity of the external water (41) originating from the external reservoir (40), using the xCO2 obtained based on the reference water with known alkalinity and storing this information in the control unit (70).

Step of transferring the external water (41) from the measurement reservoir (10) to the transfer reservoir (20) by means of the activation by the control unit (70) of the second pumping device (52) in order to completely transfer the external water from the measurement reservoir (10) to the transfer reservoir (20).

Step of transferring the external water from the transfer reservoir (20) to the external reservoir (40) by means of the activation by the control unit (70) of the third pumping device (53) in order to completely transfer the external water (41) from the transfer reservoir (20) to the external reservoir (40).

To finish the reading process of the mensuration device (1), the step of transferring the reference water from the reference reservoir (30) to the measurement reservoir (10) is carried out by means of the activation by the control unit (70) of the first pumping device (51) in order to transfer the reference water from the reference reservoir (30) to the measurement reservoir (10), keeping the pH sensor submerged.

The reading process described above concludes obtaining an unknown alkalinity of the external water (41) present in the external reservoir (40) based on a reference water with known alkalinity using as a basis the CO2 equilibrium and CO2SYS mathematical model.

Preferably, the proposed process further comprises a process of correcting the alkalinity of the external liquid present in the external reservoir, which may occur with each process of reading the alkalinity of said liquid from the external reservoir (40). In this case, the control unit (70) of the mensuration device (1) is able to perform the following steps.

If the alkalinity of the external water (41) present in the external reservoir (40) is below the target desired by the user, the second port (92) is activated, which may be connected to a calcium reactor, in order to increase the alkalinity of the external water present in the external reservoir (40).

In case the alkalinity of the external water (41) present in the external reservoir is above the target desired by the user, the device connected to the second port (92), generally used for a calcium reactor, is turned off in order to decrease the alkalinity of the external reservoir (40).

Advantageously, with the use of the proposed mensuration device (1) and process that are based on the mathematical model CO2SYS, which calculates the properties of water in marine carbonate systems from two parameters, it is possible to carry out a continuous, accurate and reliable process of measuring and correcting the alkalinity of aquarium water, without the frequent use of chemical reagents.

Therefore, it is noted that the proposed mensuration device (1) and process allow the CO2SYS method to be properly operated by means of the components and steps that make up said device (1) and process.

Preferred or alternate embodiment described herein are not to be construed as limiting this invention to the structural forms, and constructive variations may be equivalent without, however, departing from the scope of protection of the invention.

1-12. (canceled)

13. A device for measuring the alkalinity of a liquid present in an external reservoir, comprising
a reference reservoir (30) and a transfer reservoir (20) cooperating with a measurement reservoir (10), wherein at least one pH sensor (12) being arranged inside said measurement reservoir (10);
a first pumping device (51) cable of transferring a liquid between the reference reservoir (30) and the measurement reservoir (10), a second pumping device (52) capable of transferring a liquid between the transfer reservoir (20) and the measurement reservoir (10), and a third pumping device (53) capable of transferring a liquid between the transfer reservoir (20) and an external reservoir (40) to the mensuration device (1); and
an air pump (60) capable of injecting air into the interior of the metering reservoir (10) and into the interior of the transfer reservoir (20),
characterized in that it comprises a control unit (70) cooperating:
with the pH sensor (12) of the measurement reservoir (10), in order to receive and store the signals from said pH sensor (12); and
with the pumping devices (51,52,53) and the air pump (60), in order to enable the actuation of said pumping devices (51,52,53) and of said air pump (60) from pre-programmed configurations,
wherein the control unit (70) is able to determine the alkalinity of an external liquid (41) present in the external reservoir (40) by using a CO2SYS method in conjunction with information previously stored in said control unit (70).

14. The device, according to claim 13, characterized in that it comprises a level sensor (11,21,31) arranged inside each of the reservoirs (10,20,30), and that the control unit (70) is able to cooperate in order to receive and store the signals from said level sensors (11,21,31).

15. The device, according to claim 14, characterized in that it comprises a structure (80) on which the reservoirs (10,20,30), the pumping devices (51,52,53), the air pump (60) and the control unit (70) are mounted.

16. The device, according to claim 15, characterized in that the measurement reservoir (10) and that the transfer reservoir (20) are arranged inside the reference reservoir (30).

17. The device, according to claim 16, characterized in that it comprises a first porous stone (611) and a second porous stone (621) cooperating with the air pump (60), said first porous stone (611) being arranged inside the measurement reservoir (10) and said second porous stone (621) being arranged inside the transfer reservoir (20).

18. The device, according to claim 17, characterized in that it comprises at least one connection port (90) cooperating with the control unit (70).

19. The device, according to claim 18, characterized in that it comprises a correction device capable of depositing correction products inside the external reservoir (40) upon activation of the control unit (70).

20. A process for measuring the alkalinity of a liquid present in an external reservoir, comprising a mensuration device (1) of alkalinity cooperating with said external reservoir (40) in which said liquid (41) is, characterized in that said mensuration device (1) comprises a control unit (70) capable of performing;
a calibration process, in which the alkalinity of a reference liquid present in a reference reservoir (30) of the mensuration device (1) is determined; and
a reading process, in which the alkalinity of a liquid present in an external reservoir (40) to the mensuration device (1) is determined by using a CO2SYS method in conjunction with information previously stored in said control unit (70).

21. The process, according to claim 20, characterized in that the determination of the alkalinity of the reference liquid present in the reference reservoir (30) of the mensuration device (1), in the calibration process, occurs through the use of a CO2SYS method in conjunction with information previously stored in the control unit (70).

22. The process, according to claim 21, wherein the calibration process comprises the steps of;
Transferring liquid with unknown alkalinity from a reference reservoir (30) to a measurement reservoir (10);
Transferring liquid with known alkalinity from an external reservoir (40) to a transfer reservoir (20);
Measure pH of the liquid with unknown alkalinity present in the measurement reservoir (10) and store this information in the control unit (70);
Inject air into the liquid present in the measurement reservoir (10) and the liquid present in the transfer reservoir (20) for a sufficient time to stabilize the relative pH, and store the pH information of the liquid with unknown alkalinity that is in the measurement reservoir (10);
Transfer liquid with unknown alkalinity from the measurement reservoir (10) to the reference reservoir (30);
Transferring liquid with known alkalinity from the transfer reservoir (20) to the measurement reservoir (10) and injecting air into the liquid with known alkalinity from said measurement reservoir (10) until stabilizing the pH and storing the pH information of the liquid with known alkalinity in the control unit (70);
Use the CO2SYS method to calculate the xCO2 for the air inside the reservoirs (10,20,30);
Use the previously calculated xCO2 together with the relative pH of the reference liquid present in the reference reservoir (30) to calculate the unknown alkalinity of said reference liquid by means of the CO2SYS method and store this information in the control unit (70).

23. The process, according to claim 22, characterized in that the reading process comprises the steps of;
Transfer liquid with known alkalinity from the reference reservoir (30) to the measurement reservoir (10);
Transfer liquid (41) with unknown alkalinity from the external reservoir (40) to the transfer reservoir (20);
Measure relative pH of the liquid with known alkalinity present in the measurement reservoir (10) and store this information in the control unit (70);
Inject air into the liquid with known alkalinity present in the measurement reservoir (10) and into the liquid with unknown alkalinity present in the transfer reservoir (20) for sufficient time to stabilize the relative pH, and then store the pH information of the liquid with known alkalinity that is in the measurement reservoir (10);
Transfer liquid with known alkalinity from the measurement reservoir (10) to the reference reservoir (30);
Transferring liquid with unknown alkalinity from the transfer reservoir (20) to the measurement reservoir (10) and injecting air into the liquid with unknown alkalinity from said measurement reservoir (10) until stabilizing the pH and storing the pH information of the liquid with unknown alkalinity in the control unit (70);
Use of the CO2SYS method to obtain the alkalinity of the liquid from the external reservoir (40), using the xCO2 obtained based on the reference liquid with known alkalinity, and store this information in the control unit (70).

24. The process, according to claim 23, characterized in that it comprises a process of correcting the alkalinity of the external liquid (41) present in the external reservoir (40).
